(19) United States
(12) Patent Application Publication
(10) Pub. No.: US 2021/0267513 A1
(43) Pub. Date: Sep. 2, 2021

(54) METHOD FOR ANALYZING DYNAMIC CHARACTERISTIC OF EEG FUNCTIONAL CONNECTIVITY RELATED TO DRIVING FATIGUE

(71) Applicant: WUYI University, Guangdong (CN)

(72) Inventors: Hongtao Wang, Guangdong (CN); Xucheng LIU, Guangdong (CN); Ting Li, Guangdong (CN); Cong TANG, Guangdong (CN); Zi'an PEI, Guangdong (CN); Hongwei YUE, Guangdong (CN); Peng CHEN, Guangdong (CN); Tao Xu, Guangdong (CN)

(21) Appl. No.: 16/975,683

(22) PCT Filed: Oct. 8, 2019
(86) PCT No.: PCT/CN2019/109853

§ 371 Date: Aug. 25, 2020

(30) Foreign Application Priority Data
Sep. 11, 2019 (CN) 201910859394.9

(51) Int. Cl. A61B 5/16; A61B 5/374; A61B 5/18; A61B 5/00; A61B 5/398

(52) U.S. Cl. CPC A61B 5/165; A61B 5/374; A61B 2503/22; A61B 5/726; A61B 5/398; A61B 5/18

(57) ABSTRACT

Disclosed is a method for analyzing dynamic characteristics of EEG functional connectivity related to driving fatigue including: using independent component analysis and wavelet packet transformation to preprocess EEG data; constructing the preprocessed EEG data into a temporal brain network with dynamic characteristics based on a sliding window method; measuring a spatiotemporal topology of the temporal brain network based on a temporal efficiency analysis framework; and performing statistical analysis on the spatiotemporal topology of the temporal brain network to obtain a correlation between behaviors related to driving fatigue and dynamic characteristics of the temporal brain network.

### FIELD

The disclosure relates to the field of driving fatigue analysis technologies, and more particularly, to a method for analyzing dynamic characteristics of EEG functional connectivity related to driving fatigue.

### BACKGROUND

Driving fatigue has long been considered as one of major causes of fatal accidents in the world. There is evidence showing that 15% to 20% of fatal traffic accidents are related to the driving fatigue. Therefore, researchers have made great efforts in recent years in a new field of neuroergonomics to understand the neurobiological basis of the driving fatigue, with a purpose of developing an applicable automatic detection technology and reducing fatigue-related traffic accidents in the real world.

At present, it is an effective method to collect experimental data based on electroencephalogram (EEG), extract features, and then construct a fatigue-related EEG functional connectivity (FC) architecture. In the past, FC in fatigue research was a static connectivity, that is, a representative brain network was constructed on a temporal scale of several minutes under fatigue. However, static network research lacks a more critical dynamic characteristic of information transmission function reorganization among brain regions related to driving fatigue on a fine temporal scale. Therefore, experimental results under a static FC architecture have certain limitations.

### SUMMARY

In order to solve the problems above, the disclosure is intended to provide a method for analyzing dynamic characteristics of EEG functional connectivity related to driving fatigue, in which a dynamic FC analysis framework is applied to study driving fatigue, so as to obtain a more critical dynamic characteristic of information transmission function reorganization among brain regions related to driving fatigue on a fine temporal scale and obtain a higher recognition accuracy. The technical solutions used in the present invention to solve the problems thereof are as follows.

There is provided a method for analyzing dynamic characteristics of EEG functional connectivity related to driving fatigue including:

using independent component analysis and wavelet packet transformation to preprocess EEG data;

constructing the preprocessed EEG data into a temporal brain network with dynamic characteristics based on a sliding window method;

measuring a spatiotemporal topology of the temporal brain network based on a temporal efficiency analysis framework; and

performing statistical analysis on the spatiotemporal topology of the temporal brain network to obtain a correlation between behaviors related to driving fatigue and dynamic characteristics of the temporal brain network, wherein the correlation includes a spatiotemporal global efficiency, a spatiotemporal local efficiency and a spatiotemporal closeness centrality.

Further, the using independent component analysis and wavelet packet transformation to preprocess the EEG data includes:

collecting blink artifact data including horizontal electrooculogram HEOG data and vertical electrooculogram VEOG data;

using the independent component analysis to search and delete a component in the EEG data highly correlated with the blink artifact data;

removing a baseline of screened EEG data;

using the wavelet packet transformation to decompose the EEG data into three standard frequency bands which are respectively frequency band α, frequency band β and frequency band θ; and

dividing the EEG data into alert-state data and fatigue-state data according to a test time.

Further, the constructing the preprocessed EEG data into a temporal brain network with dynamic characteristics based on a sliding window method includes:

expressing the preprocessed EEG data into static networks, wherein each of the static networks is a binary N×N matrix, and N represents a number of electrodes of an EEG cap;

selecting a suitable window length and a suitable step size, and sequentially traversing a whole temporal sequence by a sliding window, wherein a length of the temporal sequence is an experimental duration for collecting the EEG data;

estimating a PLI value of functional connectivity using a phase lag index in each of the static networks;

using a sparsity method to set the PLI value greater than a threshold value to be 1, and set the PLI value smaller than the threshold value to be 0, thus forming a binary neighboring network which is used as a snapshot of the temporal brain network; and

arranging the static networks according to the temporal sequence to form the temporal brain network with dynamic characteristics.

Further, the measuring a spatiotemporal topology of the temporal brain network based on a temporal efficiency analysis framework includes:

calculating a temporal distance of a pair of nodes on a temporal scale, wherein the temporal distance represents a minimum number of temporal windows which are defined to be passed through by a spatiotemporal path;

calculating a spatiotemporal global efficiency;

calculating a spatiotemporal local efficiency; and

using a spatiotemporal closeness centrality to evaluate spatiotemporal characteristics of a node-level temporal brain network.

There is provided a device for analyzing dynamic characteristics of EEG functional connectivity related to driving fatigue includes:

a preprocessing unit configured to use independent component analysis and wavelet packet transformation to preprocess EEG data;

a construction unit configured to construct the preprocessed EEG data into a temporal brain network with dynamic characteristics based on a sliding window method;

a spatiotemporal topology calculation unit configured to measure a spatiotemporal topology of the temporal brain network based on a temporal efficiency analysis framework; and

a statistical analysis unit configured to perform statistical analysis on the spatiotemporal topology of the temporal brain network to obtain a correlation between behaviors related to driving fatigue and dynamic characteristics of the temporal brain network, wherein the correlation includes a spatiotemporal global efficiency, a spatiotemporal local efficiency and a spatiotemporal closeness centrality.

Further, the preprocessing unit includes:

a blink artifact data collection unit configured to collect blink artifact data including horizontal electrooculogram HEOG data and vertical electrooculogram VEOG data;

a screening unit configured to use the independent component analysis to search and delete a component in the EEG data highly correlated with the blink artifact data;

a baseline removal unit configured to remove a baseline of screened EEG data;

a decomposition unit configured to use the wavelet packet transformation to decompose the EEG data into three standard frequency bands which are respectively frequency band α, frequency band β and frequency band θ; and

a data division unit configured to divide the EEG data into alert-state data and fatigue-state data according to a test time.

Further, the construction unit includes:

a matrix construction unit configured to express the preprocessed EEG data into static networks, wherein the static network is a binary N×N matrix, and N represents a number of electrodes of an EEG cap;

a sliding window processing unit configured to select a suitable window length and a suitable step size, and sequentially traverse a whole temporal sequence by a sliding window, wherein a length of the temporal sequence is an experimental duration for collecting the EEG data; and

a PLI calculation unit configured to estimate a PLI value of functional connectivity using a phase lag index in each of the static networks;

a binarization calculation unit configured to use a sparsity method to set the PLI value greater than a threshold value to be 1, and set the PLI value smaller than the threshold value to be 0, thus forming a binary neighboring network which is used as a snapshot of the temporal brain network;

a sparsity calculation unit configured to select a suitable sparsity and a suitable interval and maintain required functional connectivity in each static network by a sparsity method; and

dynamic characteristics construction unit configured to arrange the static networks according to the temporal sequence to form the temporal brain network with dynamic characteristics.

Further, the spatiotemporal topology calculation unit includes:

a temporal distance calculation unit configured to calculate a temporal distance of a pair of nodes on a temporal scale, wherein the temporal distance represents a minimum number of temporal windows which are defined to be passed through by a spatiotemporal path;

a spatiotemporal global efficiency calculation unit configured to calculate a spatiotemporal global efficiency;

a spatiotemporal local efficiency calculation unit configured to calculate a spatiotemporal local efficiency; and

a spatiotemporal characteristic evaluation unit configured to use the spatiotemporal closeness centrality to evaluate spatiotemporal characteristics of a node-level temporal brain network.

There is provided an apparatus for analyzing dynamic characteristics of EEG functional connectivity related to driving fatigue including at least one control processor and a memory communicated with the at least one control processor, the memory having instructions executable by the at least one control processor stored thereon, and the instructions, when executed by the at least one control processor, enable the at least one control processor to execute the method for analyzing dynamic characteristic of EEG functional connectivity related to driving fatigue as described above.

According to a fourth aspect of the disclosure, there is provided a computer-readable storage medium with computer-executable instructions stored thereon, and the computer-executable instructions, when executed, enable a computer to execute the method for analyzing dynamic characteristic of EEG functional connectivity related to driving fatigue as described above.

In the disclosure, the temporal brain network with dynamic characteristics is constructed by introducing the temporal characteristic into the static network of the driving fatigue, a spatiotemporal recombination rule of the temporal brain network during the driving fatigue can be obtained through statistical analysis. Compared with static FC in current driving fatigue study, the analysis method of the disclosure has a more accurate analysis result, and is beneficial for revealing a more critical dynamic characteristic of information transmission function reorganization among brain regions related to driving fatigue on a fine temporal scale.

### BRIEF DESCRIPTION OF THE DRAWINGS

The disclosure is further described hereinafter with reference to the accompanying drawings and the embodiments.

FIG. 1 is a flowchart of an overall method according to an embodiment of the disclosure;

FIG. 2 is a flowchart of a preprocessing method according to an embodiment of the disclosure:

FIG. 3 is a flowchart of constructing a temporal brain network with dynamic characteristics according to an embodiment of the disclosure;

FIG. 4 is a flowchart of measuring a spatiotemporal topology of the temporal brain network according to an embodiment of the disclosure;

FIG. 5 is a schematic diagram of units in a device according to an embodiment of the disclosure; and

FIG. 6 is a schematic diagram illustrating connection in an apparatus according to an embodiment of the disclosure.

### DETAILED DESCRIPTION OF THE EMBODIMENTS

To make the objectives, technical solutions, and advantages of the disclosure clearer, the disclosure is further described in detail hereinafter with reference to the accompanying drawings and the embodiments. It should be understood that the specific embodiments described herein are merely used to explain the disclosure, but are not used to limit the disclosure. It should be noted that if there is no conflict, various features in the embodiments of the disclosure can be combined with each other, which all fall within the protection scope of the disclosure.

It should be noted that if there is no conflict, various features in the embodiments of the disclosure can be combined with each other, which all fall within the protection scope of the disclosure. In addition, although separation for functional modules is performed in a schematic diagram of a device, and a logical sequence is shown in a flowchart, in some cases, steps shown or described may be performed in a sequence different from that for module separation or that shown in the flowchart.

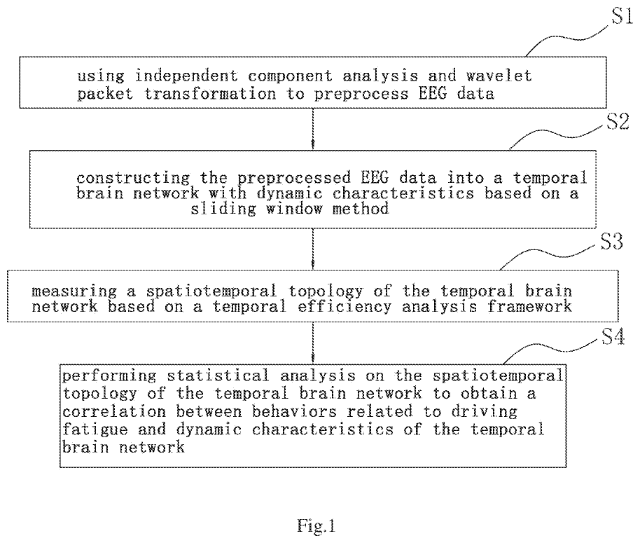

With reference to FIG. 1, in an embodiment of the disclosure, there is provided a method for analyzing dynamic characteristics of EEG functional connectivity related to driving fatigue including:

S1: using independent component analysis and wavelet packet transformation to preprocess EEG data;

S2: constructing the preprocessed EEG data into a temporal brain network with dynamic characteristics based on a sliding window method:

S3: measuring a spatiotemporal topology of the temporal brain network based on a temporal efficiency analysis framework; and

S4: performing statistical analysis on the spatiotemporal topology of the temporal brain network to obtain a correlation between behaviors related to driving fatigue and dynamic characteristics of the temporal brain network, wherein the correlation includes a spatiotemporal global efficiency, a spatiotemporal local efficiency and a spatiotemporal closeness centrality.

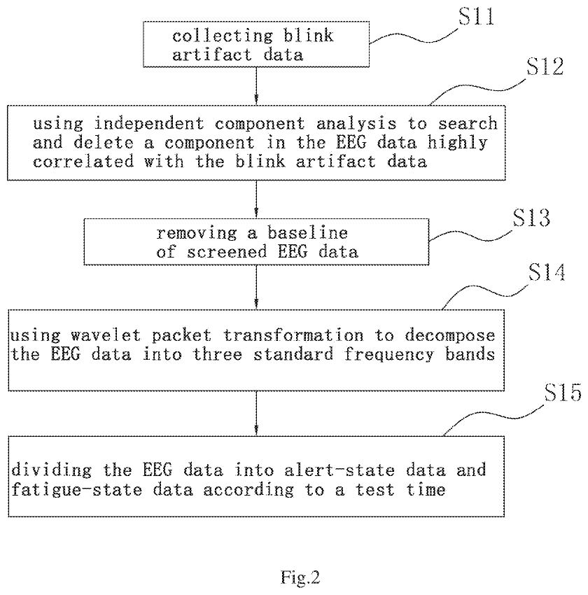

With reference to FIG. 2, the step S1 includes:

S11: collecting blink artifact data, wherein the blink artifact data includes horizontal electrooculogram HEOG data and vertical electrooculogram VEOG data;

S12: using the independent component analysis to search and delete a component in the EEG data highly correlated with the blink artifact data;

S13: removing a baseline of screened EEG data;

S14: using the wavelet packet transformation to decompose the EEG data into three standard frequency bands which are respectively frequency band α, frequency band β and frequency band θ: and

S15: dividing the EEG data into alert-state data and fatigue-state data according to a test time.

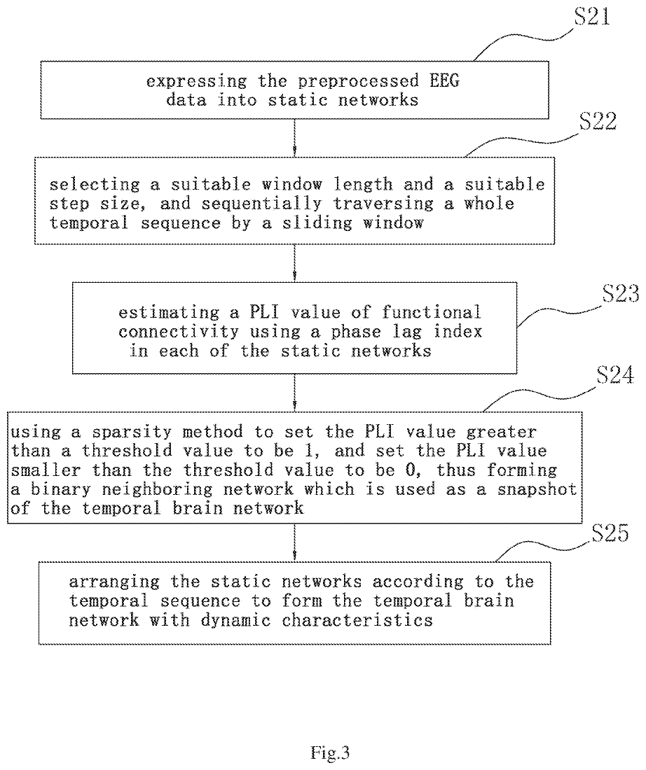

With reference to FIG. 3, the step S2 includes:

S21: expressing the preprocessed EEG data into static networks, wherein each of the static networks is a binary N×N matrix, and N represents a number of electrodes of an EEG cap;

S22: selecting a suitable window length and a suitable step size, and sequentially traversing a whole temporal sequence by a sliding window, wherein a length of the temporal sequence is an experimental duration for collecting the EEG data;

S23: estimating a PLI value of functional connectivity using a phase lag index in each of the static networks;

S24: using a sparsity method, by a binarization calculation unit, to set the PLI value greater than a threshold value to be 1, and set the PLI value smaller than the threshold value to be 0, thus forming a binary neighboring network which is used as a snapshot of the temporal brain network; and

S25: arranging the static networks according to the temporal sequence to form the temporal brain network with the dynamic characteristics.

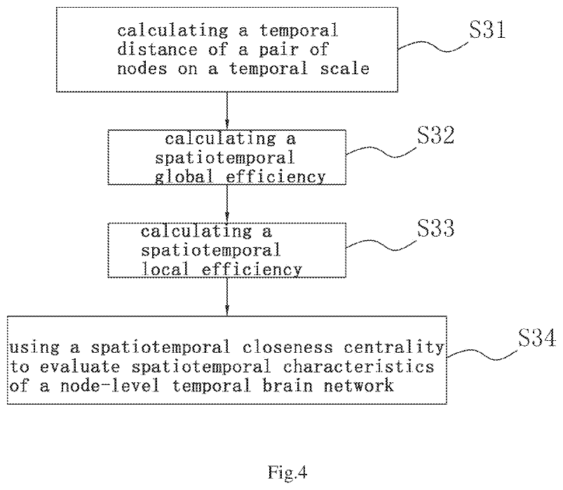

With reference to FIG. 4, the step S3 includes:

S31: calculating a temporal distance of a pair of nodes on a temporal scale, wherein the temporal distance represents a minimum number of temporal windows which are defined to be passed through by a spatiotemporal path;

S32: calculating the spatiotemporal global efficiency;

S33: calculating the spatiotemporal local efficiency; and

S34: using a spatiotemporal closeness centrality to evaluate spatiotemporal characteristics of a node-level temporal brain network.

The analysis method of the disclosure is described in detail according to an overall process as follows.

In the past, connectivity (FC) in study of driving fatigue was static, that is, a representative brain network was constructed on a temporal scale of several minutes under a fatigue state. Since a key component of the driving fatigue is task time itself, which emphasizes the accumulated characteristic of a process for a brain to adjust a decline change of a fatigue-related performance, study of a static brain network lacks a more critical dynamic characteristic of information transmission function reorganization among brain regions related to driving fatigue on a fine temporal scale.

In an experiment according to an embodiment of the disclosure there is provided that, a subject performs simulated driving for 90 minutes, and a virtual guide vehicle which is travelling is arranged in front of the subject, the virtual guide vehicle generates braking signals at random intervals, and the subject is required to respond to the braking signals through braking, so as to keep a safe distance. A time interval from a braking command generated by the virtual guide vehicle to a braking operation performed by the subject is considered as a reaction time (RT), and meanwhile, a speed variation (SV) of the vehicle is also collected as a quantitative index for evaluating a behavior of the subject.

A data collection method is that the subject wears a 24-channel wireless EEG cap with an improved 10-20 international electrodes placement system (HD-72, Cognionics, Inc., USA) to record EEG data at 250 Hz, reference electrodes are right and left mastoids, and EEG signal is filtered by a bandpass filter (between 2 Hz and 100 Hz). A blink artifact is simultaneously recorded by electrodes placed at outer corners of eyes (horizontal electrooculogram, HEOG), and above and below a right eye (vertical electrooculogram. VEOG).

Data Preprocessing:

Independent component analysis (ICA) is used to search and delete a component highly related to recorded blink artifact data, a baseline is removed from whole experimental data, and processed data is decomposed into three standard frequency bands by wavelet packet transformation (WPT) which are respectively θ (0.3 Hz to 7 Hz), α (8 Hz to 13 Hz) and β (14 Hz to 30 Hz). In the embodiment of the disclosure, a Daubechies wavelet with db4 and a decomposition level 6 is used in the wavelet packet transformation to extract EEG information.

Construction of Functional Connectivity and Temporal Brain Network:

A dynamic analysis framework of a brain network uses an optimal sliding window to characterize sequential static networks. In each static network, a phase lag index (PLI) is used to estimate the FC, because it is advantageous in minimizing influence of common source signal and volume conduction, a PLI value of the FC is between 0 and 1, and the larger the value is, the stronger the connectivity is. Then, matrix thresholding is conducted to obtain a binary neighboring network by a common sparsity method with a sparsity ranging from 5% to 15% and an interval of 1%, which is equivalent to comparing the PLI value of the FC with a threshold value. The PLI value of the FC greater than the threshold value is set to be 1, and the PLI value of the FC smaller than the threshold value is set to be 0, thus obtaining the binary neighboring network which is used as a snapshot of a temporal brain network. Therefore, the temporal brain network G={Gt} can be expressed by individual static networks Gt arranged according to a temporal sequence t, wherein t represents a positive integer. Each static network is a binary N×N matrix, a number of the FCs is the same as that of the static networks, and in the experiment according to the embodiment of the disclosure. N is 24.

In the embodiment of the disclosure, window length may be selected from 3 seconds to 6 seconds, and step size may be selected from 2 seconds to 4 seconds. In order to balance dynamics of signals and quality of connectivity estimation and reduce computational complexity, the window length is selected to be 4 seconds, and the step size is selected to be 4 seconds. Most alert and fatigue states are determined by statistically comparing the behaviors of the subject in terms of the response time and the speed variation, and the first 5 minutes and the last 5 minutes in the EEG data are finally selected as analysis data, respectively corresponding to the most alert and fatigue states, so that the temporal step size of each window within 5 minutes is T=75.

### Spatiotemporal Topology of the Temporal Brain Network:

After the temporal brain network with the dynamic characteristics is constructed according to the method above, a temporal distance of a pair of nodes needs to be calculated on a temporal scale, the temporal distance is defined as a minimum number of temporal windows passed through by a spatiotemporal path, it is worth noting that a time-related path is a measurement of space and time domains, and the temporal distance is characterized by the time domain. Therefore, the temporal distance is a positive integer and ranges between 1 and T.

In order to quantitatively reveal dynamic reorganization of a brain during the driving fatigue, a spatiotemporal efficiency analysis framework is adopted to measure the spatiotemporal topology of the temporal brain network. Conceptually, spatiotemporal efficiency measures interaction and information transmission functions among all nodes in a dynamic system, spatiotemporal global efficiency captures dynamics of the entire network, and information flow capability is propagated throughout a life cycle. A calculation method is as follows.

Assuming the spatiotemporal global efficiency is expressed as E_{glob}^{t}(G), then

$${E}_{glob}^{t} ( G ) = \frac{1}{T} {\sum }_{t ∈ 1 , 2 , 3 , … , T} {E}_{glob}^{t} ( G , t )$$

wherein G is a spatiotemporal network with a mathematical structure N×N×T, and E_{glob}^{t}(G, t) is efficiency of the spatiotemporal global efficiency at a time t, and t is a positive integer not greater than T.

Therefore.

$${E}_{glob}^{t} ( G , t ) = \frac{1}{N ( N - 1 )} {\sum }_{i \neq j ∈ 1 , 2 , 3 , … , N} \frac{1}{{{τ}_{i}}_{\to j} ( t )}$$

E_{glob}^{t}(G, t) ranges between 0 and 1, E_{glob}^{t}(G, t)=1=1 represents that all nodes are connected in the snapshot of the spatiotemporal brain network, and τ_{i→j}(t) represents a temporal distance from i to j at a time t. A spatiotemporal local efficiency measures an overall elasticity of the dynamic network to randomly remove nodes within a local range.

If the spatiotemporal local efficiency is expressed as E_{loc}^{t}(G), then

$${E}_{loc}^{t} ( G ) = \frac{1}{N} {\sum }_{i ∈ 1 , 2 , 3 , … , N} [ \frac{1}{T} {\sum }_{i ∈ 1 , 2 , 3 , … , N} ( {E}_{glob}^{t} ( G ( i , t ) , t ) ) ]$$

wherein G(i, t) is a spatiotemporal sub-network including all neighbors of a node i at a time t. E_{loc}^{t}(G) ranges between 0 and 1, and is an index of the dynamic network for measuring information transmission capability on a local scale.

A spatiotemporal closeness centrality is used to evaluate spatiotemporal characteristics of a node-level temporal brain network, and the centrality measures a capability C_{c}(i, t) of a node i reaching other nodes, which is expressed as:

$${C}_{c} ( i , t ) = \frac{1}{N} {\sum }_{i \neq j} \frac{1}{{τ}_{i \to j} ( t )}$$

The spatiotemporal closeness centrality also represents importance of the node in the entire temporal network. An integral spatiotemporal closeness centrality represents an area under the curve of the node i in an entire sparsity range.

Reference Network:

Considering a richness of complex structures in dynamic FC, a certain processing method needs to be adopted to reveal characteristics and advantages of the dynamic brain network compared with a reference network. The reference network increases a randomness of the dynamic brain network and presents different information transmission efficiencies. Calculation of the reference network is beneficial for revealing a neural mechanism of the dynamic FC with different topologies. In the embodiment of the disclosure, a two-step randomization method is used: random edge (RE) and random connectivity (RC). The RE method randomly reconnects all edges in the dynamic network under certain constraints, which destroys a topological structure of the dynamic brain network. The RC method randomly redistributes all connectivities in the network, which eliminates distribution of connectivity number of each edge. Application of the two methods destroys a main structure of the dynamic network. Definition of a small-world property in the static network is expanded to the temporal brain network with the dynamic characteristics, and the temporal brain network with the dynamic characteristics is spatiotemporally small-world if the following definition is met:

E_{loc}^{t}/E_{loc_rand}^{t}<<1

or

E_{loc}^{t}/E_{loc_rand}^{t}≈1

Spatiotemporal efficiency of the reference network of each subject is an average value of the spatiotemporal reference network generated with 50 iterations in two mental states (alert and fatigue states).

Statistical Analysis:

In order to study an influence of the driving fatigue on a driver's control ability, one-way repeated measures ANOVA is used to calculate the response time and the speed variation during a whole simulated driving task, that is, the one-way ANOVA is used to search key differences in the spatiotemporal global efficiency, the spatiotemporal local efficiency and the spatiotemporal closeness centrality between the alert and fatigue states. Therefore, Pearson correlation is performed to evaluate correlations between fatigue-related behaviors and characteristics of the dynamic brain network. These correlations include an integrated spatiotemporal global efficiency, an integrated spatiotemporal local efficiency and an integrated spatiotemporal closeness centrality. The Pearson correlation evaluation is expressed by p, and is considered to be significantly correlated when p<0.05. Finally, correction of multiple comparisons of regional characteristics is performed through a fault detection rate (FDR) of q=0.05.

Result Analysis:

Values of the reaction time (RT) and the speed variation (SV) in the first 5 minutes and the last 5 minutes are respectively expressed by coordinates to form a time vs. reaction time coordinate graph and a time vs. speed variation coordinate graph. It is obvious that a significant difference exists between the two states in the first 5 minutes and the last 5 minutes, thus confirming that definition of the first 5 minutes as the alert state and definition of the last 5 minutes as the fatigue state above are reasonable.

Therefore, a spatiotemporal topology of a brain activity is quantitatively estimated through spatiotemporal efficiencies in the alert and fatigue states respectively, and a frequency band vs. efficiency coordinate graph is established according to division of three standard frequency bands, so as to display the integrated spatiotemporal global efficiency and the integrated spatiotemporal local efficiency in the whole sparsity range. The temporal brain network with the dynamic characteristics shows a prominent spatiotemporally small-world architecture in all three frequency bands: a standard deviation is used to represent a degree of dispersion of brain connectivity distribution across the subjects, this discovery may indicate a significant difference among the subjects in reorganization of the dynamic FC under the fatigue state, it can be seen from calculation that standard deviations of the spatiotemporal global efficiency and the spatiotemporal local efficiency under the fatigue state are significantly higher than those under the alert state, and the result is consistent with a theory that an individual has a characteristic feature, which tends to have an accompanying vulnerability of biological matrix which changes over time.

Spatiotemporal Closeness Centrality:

A comprehensive spatiotemporal closeness centrality of 24 nodes is calculated to evaluate a spatiotemporal characteristic of a node of the dynamic FC, it can be seen from calculation that the comprehensive spatiotemporal closeness centrality of the 24 nodes in the fatigue state is generally lower than that in the alert state, and meanwhile, it can be seen from brain regions corresponding to collected EEG data that nodes in frontal and parietal lobes usually show great difference between the alert and fatigue states, that is, a calculation result meets p<0.05.

Relationship Between Behavior and Network Property:

A relationship between behavior measurements ΔRT and ΔSV and the dynamic characteristic ΔE of the temporal brain network is studied by bivariate correlation analysis:

ΔRT=RT_{fatigue}−RTₐₗₑᵣₜ

ΔSV=SV_{fatigue}−SVₐₗₑᵣₜ

ΔE=E_{fatigue}−Eₐₗₑᵣₜ

According to analysis of a correlation between the behavior measurements ΔRT and ΔSV and the dynamic characteristic ΔE in three standard frequency bands, a correlation result between frequency band and efficiency can be obtained. For a node property, only nodes showing a significant fatigue correlation difference in each frequency band are selected for correlation testing.

The embodiment of the disclosure introduces a dynamic FC analysis framework and provides a method for analyzing spatiotemporal reorganization of the dynamic network during study of the driving fatigue. In the analysis method, a small-world property existing in a static brain network is expanded to a dynamic system, which, in combination with introduction of a temporal factor to the FC and taking a graph theory property (global efficiency and local efficiency) as a characteristic, obtains a higher identification accuracy than a traditional analysis method, thus proving a feasibility of the spatiotemporal architecture and spatiotemporal efficiency method in driving fatigue detection.

According to an embodiment of the disclosure, there is further provided a device for analyzing dynamic characteristics of EEG functional connectivity related to driving fatigue, and the device 1000 for analyzing dynamic characteristics of EEG functional connectivity related to driving fatigue includes but not limited to: a preprocessing unit 1100, a construction unit 1200, a spatiotemporal topology calculation unit 1300 and a statistical analysis unit 1400.

The preprocessing unit 1100 is configured to use independent component analysis and wavelet packet transformation to preprocess EEG data.

The construction unit 1200 is configured to construct the preprocessed EEG data into a temporal brain network with dynamic characteristics based on a sliding window method.

The spatiotemporal topology calculation unit 1300 is configured to measure the spatiotemporal topology of the temporal brain network based on a temporal efficiency analysis framework.

The statistical analysis unit 1400 is configured to perform statistical analysis on the spatiotemporal topology of the temporal brain network to obtain a correlation between behaviors related to driving fatigue and dynamic characteristics of the temporal brain network, wherein the correlation includes a spatiotemporal global efficiency, a spatiotemporal local efficiency and a spatiotemporal closeness centrality.

It shall be noted that, since the device for analyzing dynamic characteristic of EEG functional connectivity related to driving fatigue in this embodiment and the method for analyzing dynamic characteristics of EEG functional connectivity related to driving fatigue above are based on the same inventive concept, the corresponding contents in the method embodiment are also applicable to the device embodiment and will not be described in detail here.

The disclosure further provides an apparatus for analyzing dynamic characteristics of EEG functional connectivity related to driving fatigue, and the apparatus 2000 for analyzing dynamic characteristics of EEG functional connectivity related to driving fatigue may be any type of intelligent terminal, such as mobile phone, tablet computer, personal computer, etc.

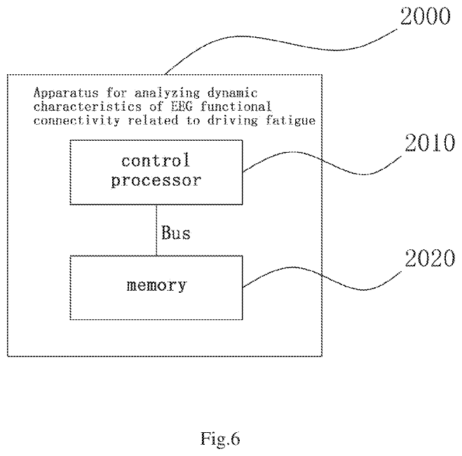

Specifically, the apparatus 2000 for analyzing dynamic characteristics of EEG functional connectivity related to driving fatigue includes one or more control processors 2010 and a memory 2020, and one control processor 2010 is taken as an example in FIG. 6.

The control processor 2010 and the memory 2020 may be connected by a bus or in other manners, and connection by the bus is taken as an example in FIG. 6.

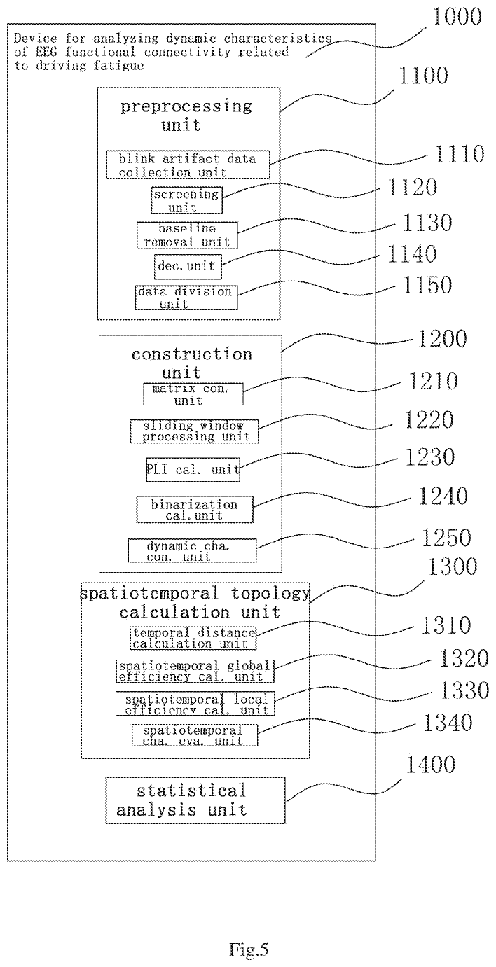

As a non-transitory computer-readable storage medium, the memory 2020 can be used to store a non-transitory software program, a non-transitory computer-executable program and a module, such as a program instruction/module corresponding to the method for analyzing dynamic characteristics of EEG functional connectivity related to driving fatigue in the embodiment of the disclosure, for example, the preprocessing unit 1100, the construction unit 1200, the spatiotemporal topology calculation unit 1300 and the statistical analysis unit 1400 shown in FIG. 5. The control processor 2010 executes various function applications and data processing of the device 1000 for analyzing dynamic characteristics of EEG functional connectivity related to driving fatigue by running the non-transitory software programs, instructions and modules stored in the memory 2020, that is, the method for analyzing dynamic characteristics of EEG functional connectivity related to driving fatigue in the method embodiment above is realized.

The memory 2020 may include a program storage region and a data storage region, wherein the program storage region can store an operating system and programs required for at least one function; and the data storage region can store data established according to use of the device 1000 for analyzing dynamic characteristics of EEG functional connectivity related to driving fatigue, etc. In addition, the memory 2020 may include a high-speed random access memory, and may also include a non-transitory memory, such as at least one disk memory apparatus, flash memory apparatus, or other non-transitory solid memory apparatus. In some embodiments, the memory 2020 may optionally include a remotely arranged memory relative to the control processor 2010, which may be connected with the apparatus 2000 for analyzing dynamic characteristics of EEG functional connectivity related to driving fatigue through a network. Examples of the network above include, but are not limited to, the Internet, intranet, local area network, mobile communication network, and combination thereof.

The one or more modules are stored in the memory 2020, and when the one or more modules are executed by the one or more control processors 2010, the method for analyzing dynamic characteristics of EEG functional connectivity related to driving fatigue in the method embodiment above is executed, for example, the method steps S1 to S4 in FIG. 1 above are executed to realize functions of the units 1100 to 1400 in FIG. 5.

The embodiment of the disclosure further provides a computer-readable storage medium with computer-executable instructions stored therein, and when the computer-executable instructions are executed by one or more control processors, for example, by the control processor 2010 in FIG. 6, the one or more control processors 2010 above are caused to execute the method for analyzing dynamic characteristics of EEG functional connectivity related to driving fatigue in the method embodiment above, for example, to execute the method steps S1 to S4 above in FIG. 1 to realize functions of the units 1100 to 1400 in FIG. 5.

The device embodiment above is only illustrative, wherein the units described as separated parts may or may not be physically separated, that is, the units may be located in one place, or may be distributed over multiple network units. Some or all of the modules may be selected according to actual needs to achieve the objectives of the solutions of the embodiments.

From the above description of the embodiments, those skilled in the art can clearly understand that various embodiments can be implemented by means of software and a general hardware platform. Those skilled in the art may understand that all or a part of the procedures of the method in the above embodiment may be implemented by instructing relevant hardware through a computer program. The program may be stored in a computer-readable storage medium, and when the program is executed, the procedures in the embodiment of the method above may be included. The storage medium may be magnetic disk, optical disk, Read Only Memory (ROM) or Random Access Memory (RAM), etc.

Those described above are merely the preferred embodiments of the disclosure described in detail, but the disclosure is not limited to the embodiments above. Those skilled in the art can make various equal deformations or replacements without departing from the concept of the disclosure, and these equal deformations or replacements shall all fall within the scope limited by the claims of the disclosure.

1: A method for analyzing dynamic characteristics of EEG functional connectivity related to driving fatigue, comprising:
using independent component analysis and wavelet packet transformation to preprocess EEG data;
constructing the preprocessed EEG data into a temporal brain network with dynamic characteristics based on a sliding window method;
measuring a spatiotemporal topology of the temporal brain network based on a temporal efficiency analysis framework; and
performing statistical analysis on the spatiotemporal topology of the temporal brain network to obtain a correlation between behaviors related to driving fatigue and dynamic characteristics of the temporal brain network, wherein the correlation comprises a spatiotemporal global efficiency, a spatiotemporal local efficiency and a spatiotemporal closeness centrality.

2: The method for analyzing dynamic characteristics of EEG functional connectivity related to driving fatigue of claim 1, wherein the using independent component analysis and wavelet packet transformation to preprocess EEG data comprises:
collecting blink artifact data comprising horizontal electrooculogram HEOG data and vertical electrooculogram VEOG data;
using independent component analysis to search and delete a component in the EEG data highly correlated with the blink artifact data;
removing a baseline of screened EEG data;
using wavelet packet transformation to decompose the EEG data into three standard frequency bands which are respectively frequency band α, frequency band β and frequency band θ; and
dividing the EEG data into alert-state data and fatigue-state data according to a test time.

3: The method for analyzing dynamic characteristics of EEG functional connectivity related to driving fatigue of claim 1, wherein the constructing the preprocessed EEG data into a temporal brain network with dynamic characteristics based on a sliding window method comprises:
expressing the preprocessed EEG data into static networks, wherein each of the static networks is a binary N×N matrix, and N represents a number of electrodes of an EEG cap;
selecting a suitable window length and a suitable step size, and sequentially traversing a whole temporal sequence by a sliding window, wherein a length of the temporal sequence is an experimental duration for collecting the EEG data;
estimating a PLI value of functional connectivity using a phase lag index in each of the static networks;
using a sparsity method to set the PLI value greater than a threshold value to be 1, and set the PLI value smaller than the threshold value to be 0, thus forming a binary neighboring network which is used as a snapshot of the temporal brain network; and
arranging the static networks according to the temporal sequence to form the temporal brain network with dynamic characteristics.

4: The method for analyzing dynamic characteristics of EEG functional connectivity related to driving fatigue of claim 1, wherein the measuring a spatiotemporal topology of the temporal brain network based on a temporal efficiency analysis framework comprises:
calculating a temporal distance of a pair of nodes on a temporal scale, wherein the temporal distance represents a minimum number of temporal windows which are defined to be passed through by a spatiotemporal path;
calculating a spatiotemporal global efficiency;
calculating a spatiotemporal local efficiency; and
using a spatiotemporal closeness centrality to evaluate spatiotemporal characteristics of a node-level temporal brain network.

5-10. (canceled)

11: An apparatus for analyzing dynamic characteristics of EEG functional connectivity related to driving fatigue, comprising:
at least one control processor, and
a memory communicated with the at least one control processor, the memory having instructions executable by the at least one control processor stored thereon, and the instructions, when executed by the at least one control processor, enable the at least one control processor to execute the steps of:
using independent component analysis and wavelet packet transformation to preprocess EEG data;
constructing the preprocessed EEG data into a temporal brain network with dynamic characteristics based on a sliding window method;
measuring a spatiotemporal topology of the temporal brain network based on a temporal efficiency analysis framework; and
performing statistical analysis on the spatiotemporal topology of the temporal brain network to obtain a correlation between behaviors related to driving fatigue and dynamic characteristics of the temporal brain network, wherein the correlation comprises a spatiotemporal global efficiency, a spatiotemporal local efficiency and a spatiotemporal closeness centrality.

12: The apparatus for analyzing dynamic characteristics of EEG functional connectivity related to driving fatigue of claim 11, wherein the using independent component analysis and wavelet packet transformation to preprocess EEG data comprises:
collecting blink artifact data comprising horizontal electrooculogram HEOG data and vertical electrooculogram VEOG data;
using independent component analysis to search and delete a component in the EEG data highly correlated with the blink artifact data;
removing a baseline of screened EEG data;
using wavelet packet transformation to decompose the EEG data into three standard frequency bands which are respectively frequency band α, frequency band β and frequency band θ; and
dividing the EEG data into alert-state data and fatigue-state data according to a test time.

13: The apparatus for analyzing dynamic characteristics of EEG functional connectivity related to driving fatigue of claim 11, wherein the constructing the preprocessed EEG data into a temporal brain network with dynamic characteristics based on a sliding window method comprises:
expressing the preprocessed EEG data into static networks, wherein each of the static networks is a binary N×N matrix, and N represents a number of electrodes of an EEG cap;
selecting a suitable window length and a suitable step size, and sequentially traversing a whole temporal sequence by a sliding window, wherein a length of the temporal sequence is an experimental duration for collecting the EEG data;
estimating a PLI value of functional connectivity using a phase lag index in each of the static networks;
using a sparsity method to set the PLI value greater than a threshold value to be 1, and set the PLI value smaller than the threshold value to be 0, thus forming a binary neighboring network which is used as a snapshot of the temporal brain network; and
arranging the static networks according to the temporal sequence to form the temporal brain network with dynamic characteristics.

14: The apparatus for analyzing dynamic characteristics of EEG functional connectivity related to driving fatigue of claim 11, wherein the measuring a spatiotemporal topology of the temporal brain network based on a temporal efficiency analysis framework comprises:
calculating a temporal distance of a pair of nodes on a temporal scale, wherein the temporal distance represents a minimum number of temporal windows which are defined to be passed through by a spatiotemporal path;
calculating a spatiotemporal global efficiency;
calculating a spatiotemporal local efficiency; and
using a spatiotemporal closeness centrality to evaluate spatiotemporal characteristics of a node-level temporal brain network.

15: A computer-readable storage medium with computer-executable instructions stored thereon, and the computer-executable instructions when executed enable a computer to execute the steps of
using independent component analysis and wavelet packet transformation to preprocess EEG data;
constructing the preprocessed EEG data into a temporal brain network with dynamic characteristics based on a sliding window method;
measuring a spatiotemporal topology of the temporal brain network based on a temporal efficiency analysis framework; and
performing statistical analysis on the spatiotemporal topology of the temporal brain network to obtain a correlation between behaviors related to driving fatigue and dynamic characteristics of the temporal brain network, wherein the correlation comprises a spatiotemporal global efficiency, a spatiotemporal local efficiency and a spatiotemporal closeness centrality.

16: The computer-readable storage medium of claim 15, wherein the using independent component analysis and wavelet packet transformation to preprocess EEG data comprises:
collecting blink artifact data comprising horizontal electrooculogram HEOG data and vertical electrooculogram VEOG data;
using independent component analysis to search and delete a component in the EEG data highly correlated with the blink artifact data;
removing a baseline of screened EEG data;
using wavelet packet transformation to decompose the EEG data into three standard frequency bands which are respectively frequency band α, frequency band β and frequency band θ; and
dividing the EEG data into alert-state data and fatigue-state data according to a test time.

17: The computer-readable storage medium of claim 15, wherein the constructing the preprocessed EEG data into a temporal brain network with dynamic characteristics based on a sliding window method comprises:
expressing the preprocessed EEG data into static networks, wherein each of the static networks is a binary N×N matrix, and N represents a number of electrodes of an EEG cap;
selecting a suitable window length and a suitable step size, and sequentially traversing a whole temporal sequence by a sliding window, wherein a length of the temporal sequence is an experimental duration for collecting the EEG data;
estimating a PLI value of functional connectivity using a phase lag index in each of the static networks;
using a sparsity method to set the PLI value greater than a threshold value to be 1, and set the PLI value smaller than the threshold value to be 0, thus forming a binary neighboring network which is used as a snapshot of the temporal brain network; and
arranging the static networks according to the temporal sequence to form the temporal brain network with dynamic characteristics.

18: The computer-readable storage medium of claim 15, wherein the measuring a spatiotemporal topology of the temporal brain network based on a temporal efficiency analysis framework comprises:
calculating a temporal distance of a pair of nodes on a temporal scale, wherein the temporal distance represents a minimum number of temporal windows which are defined to be passed through by a spatiotemporal path;
calculating a spatiotemporal global efficiency;
calculating a spatiotemporal local efficiency; and
using a spatiotemporal closeness centrality to evaluate spatiotemporal characteristics of a node-level temporal brain network.
